(19) United States
(12) Patent Application Publication
(10) Pub. No.: US 2026/0038692 A1
(43) Pub. Date: Feb. 5, 2026

(54) METHOD FOR DETERMINING A HEALTH STATE FROM INPUT DATA

(71) Applicant: Beckman Coulter, Inc., Brea, CA (US)

(72) Inventors: Maxim SHATSKY, Castro Valley, CA (US); Arnaud DEBRAINE, Arlington, VA (US)

(21) Appl. No.: 19/275,597

(22) Filed: Jul. 21, 2025

Related U.S. Application Data
(60) Provisional application No. 63/677,009, filed on Jul. 30, 2024.

(51) Int. Cl. G16H 50/30; G06N 3/0455; G06N 3/0475; G16H 50/70

(52) U.S. Cl. CPC G16H 50/30; G06N 3/0455; G06N 3/0475; G16H 50/70

(57) ABSTRACT

A computer-implemented method includes obtaining a set of first numeric vectors, each first numeric vector representative of a respective health state, the first numeric vectors having been created by using an embedding model; obtaining textual data, the textual data comprising information indicative of one or more health states of a subject; using a machine learning model for deriving, from the textual data, at least one textual element, wherein the textual element comprises information indicative of a state of the one or more health states of the subject; embedding the at least one textual element into a second numeric vector by using the embedding model; searching, among the set of first numeric vectors, a closest numeric vector that is closest to the second numeric vector; and determining the health state that is represented by the closest numeric vector as a health state indicated by the textual element.

### CROSS-REFERENCE TO RELATED APPLICATION

This application claims the benefit of U.S. Provisional Patent Application Ser. No. 63/677,009, filed Jul. 30, 2024, the entire contents of which is incorporated herein by reference.

### BACKGROUND

The present disclosure generally relates to a computer-implemented method that allows for deriving a health state from input data. Further, the present disclosure relates to a system comprising a processing system for carrying out the steps of said method, to a computer-readable medium, and a computer program product.

Present methods that automatically predict a health state of a subject based on a user input face challenges for multiple reasons, e.g. the health state of a subject may be observed in various different ways and settings, with different boundary conditions, via input by different people and in different formats leading to a significant variability of inputs to be processed. Thus, methods may suffer from reduced accuracy.

This can be mitigated by extensive pre-processing of inputs. Pre-processing of such data can be technically challenging, potentially involve manual tasks, and be rather inefficient. Moreover, such pre-processing introduces potential error sources.

This makes efficient and reliable determination of an actual health state that was meant to be represented by an input rather challenging. Conventional methods will likely often not be able to yield any meaningful results. Known AI-based methods are also challenged in terms of reliability of their predictions.

Therefore, it is an object of the present disclosure, to provide methods and systems to address these challenges, in particular to improve efficiency and reliability of evaluating a health state of a subject.

### SUMMARY

Aspects of the present disclosure relate to a computer-implemented method comprising obtaining a set of first numeric vectors, each first numeric vector representative of a respective health state, the first numeric vectors having been created by using an embedding model; obtaining textual data, the textual data comprising information indicative of one or more health states of a subject; using a machine learning model for deriving, from the textual data, at least one textual element, wherein the textual element comprises information indicative of at least one, in particular exactly one, health state of the one or more health states of the subject; embedding the at least one textual element into a second numeric vector by using the embedding model; searching, among the set of first numeric vectors, a closest numeric vector that is closest to the second numeric vector; and determining the health state that is represented by the closest numeric vector as a health state indicated by the textual element.

In particular a method is provided that leverages two machine learning models. Specifically, a data input, e.g. provided by a user or retrieved from a data source, may be processed by means of the first machine learning model and the output of the first machine learning model may be further processed by a second machine learning model, i.e., the embedding model. The output of the first machine learning model comprises textual element(s), where each textual element may be embedded in a vector by the embedding model. The method according to the present disclosure allows to determine (a) health state(s) that match(es) the data input best. Such determination can be made based on a distance between vectors representative of the health states and vector(s) representative of the output of the first machine learning model.

In the present disclosure, obtaining a set of first numeric vectors may comprise retrieving and/or receiving the first numeric vectors, e.g. from a data storage or another data source. Alternatively, or in conjunction, obtaining a set of first numeric vectors may comprise creating the first numeric vectors, e.g. by using the embedding model.

The set of first numeric vectors may comprise between 1000 and 10000, in particular between 1000 and 5000, in particular between 2000 and 4000, in particular 3000 vectors, each representative of a respective health state.

Each first numeric vector of the set of first numeric vector is representative of a respective health state. In particular, said each first numeric vector may be a vector representation of a respective representation, e.g., a respective textual representation, of the respective health state.

As an example, each first numeric vector of the set of first numeric vectors may be an embedding of the respective textual representation, e.g. a respective phrase or word, wherein the respective textual representation specifies the respective health state. For instance, if the health state is pregnancy, the respective textual representation may be “pregnancy”, “gravidity” or “Preg”.

A health state may indicate an aspect of the health of a subject. In particular, this aspect may be an aspect that can be perceived by the subject (e.g., headache, feeling cold, having a wound, shortness of breath, back pain, dizziness, abdominal pain), can be measured by a medical device (e.g., tachycardia), or can be diagnosed by a physician (e.g., diabetes). An aspect of the health of a subject may be indicative of a condition (e.g., pregnancy) or an at least partially compromised health condition (e.g., hypertension). At least a health state of the one or more health states of the subject may be a chief complaint reported by the subject about its health. For example, a health state of the one or more health states of the subject may be abdominal pain and may be reported to a physician or a nurse, which may record this complaint, e.g., by using text representing this chief complaint, e.g., “abdominal pain”, “abdo pain”, “AP” or “stomach pain”. For example, heart issue, hypertension, headache, pregnancy, drunken, abdominal pain are health states. In particular, the one or more health states may comprise one or more of: heart issue, hypertension, headache, pregnancy, drunken, abdominal pain, wound, substance abuse, shortness of breath, back pain, or dizziness.

Obtaining textual data may comprise receiving patient input data and/or nurse input data and/or physician input data via a user input, the patient input data and/or the nurse input data and/or the physician input data comprising the textual data. Alternatively or in addition, obtaining textual data may comprise retrieving the textual data from a data storage, such as a subject's Electronic Health Record (EHR) or other comparable source. The textual data may comprise structured and/or unstructured text. Alternatively or in addition, obtaining textual data may comprise obtaining the textual data from a voice recording via voice recognition.

As an example, textual data may comprise information specifying one or more health states of the subject. In particular, textual data may comprise or consist of one or more alphanumeric strings. For example, textual data may comprise one or more textual elements, each textual element comprising information indicative of a respective health state of the one or more health states of the subject. For example, textual data may consist of the following text “abdominal pain, 11 week intrauterine pregnancy”. In this case, in particular, the textual element “abdominal pain” specifies that the subject has reported an abdominal pain complaint. Moreover, the textual element “11 week intrauterine pregnancy”, specifies that the subject is pregnant.

As outlined above, the method of the present disclosure comprises deriving, from the textual data, at least one textual element. To that end, a machine learning model (such as a GPT) may be employed, as will be outlined in detail below. In particular, that machine learning model may comprise or consist of one or more foundation models, in particular one or more Generative Pre-trained transformers (GPT). For instance, that machine learning model may comprise or consist of ChatGPT. In particular, the machine learning model may be trained to parse textual data into one or more textual elements that are semantically distinct from one another. The textual data may or may not comprise the at least one textual element. Use of the machine learning model allows for deriving even textual elements that are not, as such, included in the textual data. The textual element may comprise information indicative of the one or more health states, particularly in a more concise manner, e.g. may be a single word or phrase, such as “abdominal pain”, “intrauterine pregnancy”, or “drunk”.

The textual data may be structured or unstructured text. Deriving textual elements can provide a concise and structured representation of the content of the textual data.

In particular, embedding the at least one textual element into the second numeric vector is carried out by using the embedding model. Moreover, for instance, the first numeric vectors of the set of numeric vectors are vectors that have been created by using an embedding model. Known embedding models may be used for that purpose, in particular, embedding models suitable for text inputs. Some details on the models will be provided further below.

The method of the present disclosure, as mentioned above, comprises searching, among the set of first numeric vectors, a closest numeric vector that is closest to the second numeric vector. In particular, a first numeric vector that is the closest match to the second numeric vector is found. For instance, searching the closest numeric vector may comprise determining, for each first numeric vector of the set of numeric vectors, a respective distance associated with that first numeric vector, thereby obtaining a set of distances, wherein the respective distance is the distance between that first numeric vector and the second numeric vector, thereby obtaining a set of distances. Moreover, searching the closest numeric vector may comprise determining, by using the set of distances, the closest numeric vector. For instance, determining, by using the set of distances, the closest numeric vector may comprise determining, among the set of distances, the smallest distance and determining the first numeric vector that is associated with the smallest distance as the closest numeric vector. The distance may for example be the cosine distance, the Euclidean distance, the Manhattan distance, the Chebyshev distance, the Hamming distance, or the like. In particular, the distance may be the cosine distance or the Euclidean distance.

The method according to the present disclosure comprises determining the health state that is represented by the closest numeric vector as a health state indicated by the textual element. in particular, the method determines that the information comprised in the textual element is indicative of a certain health state. Said health state is the health state representative of the first numeric vector that is closest to the second numeric vector representative of the textual element. In particular, the determination may yield one or more of the health states as output. An output may be “abdominal pain” or “pregnancy”, for example.

In particular, determining the health state that is represented by the closest numeric vector as the health state indicated by the textual element may comprise providing information specifying the health state, e.g. the respective textual representation to a user, to a database, and/or to the subject's EHR. Alternatively, or in conjunction, said information may be provided as input for other methods. For example, providing information specifying the health state to a user may comprise displaying said information to the user e.g., via a display comprised in or communicatively connected with the computing device that carries out the method according to the present disclosure. In particular, said display is accessible to the user, i.e., the user can look at the information shown on the display. For example, providing information to a database or the subject's EHR may comprise making this information available to the database or the EHR, so that that database or that EHR may access, retrieve and/or store this information in a memory that that database or that EHR may access. In particular, providing information to a database or the subject's EHR may comprise sending that information to that database or that EHR.

The present disclosure, therefore, may provide efficient reliable determination of an actual health state of the subject that was meant to be represented by an input. It allows for processing data input reliably even when said data may be incomplete and/or unstructured and/or vary in content and/or format. Although not limited thereto, the present disclosure may be of particular advantage for clinical decision support, CDS, applications.

Exemplarily, the method according to the present disclosure may comprise generating the set of first numeric vectors. In particular, generating the set of first numeric vectors comprises, for each health state of a plurality of health states, embedding a respective, particularly textual, representation of the respective health state into a respective first numeric vector of the set of first numeric vectors, said vector being representative of said each health state.

In particular, generating the first numeric vectors may be carried out at a time prior to runtime.

For instance, runtime may be considered to be the time, e.g., the time interval, when data is processed to determine the health state. Processing data to determine the health state may comprise one or more of: obtaining the textual data, obtaining the machine learning model used for determining the textual element from the textual data, using the machine learning model for deriving the textual element, obtaining the embedding model for embedding the textual element into the second numeric vector, embedding the textual element, searching the closest numeric vector, determining and optionally outputting the health state that is represented by the closest numeric vector as the health state indicated by the textual element. Processing data may optionally comprise pre-processing of data retrieved and/or received from a data source to obtain the textual data.

For example, processing data to determine the health state may comprise obtaining the textual data, using the machine learning model for deriving the textual element, embedding the textual element, searching the closest numeric vector, and determining the health state that is represented by the closest numeric vector as a health state indicated by the textual element.

Creating the first numeric vectors may be carried out once, e.g. in a one-time pre-processing step. It may alternatively be carried out more than once, e.g. to update the set of first numeric vectors with different and/or additional vectors, potentially also at runtime.

According to the present disclosure, obtaining textual data may comprise pre-processing text-based input data to obtain the textual data.

In particular, text-based input data may comprise information specifying one or more health states of the subject. In particular, the text-based input may comprise or consist of one or more alphanumeric strings. In particular, obtaining textual data may comprise receiving patient input data and/or physician input data via a user input, the patient input data and the physician input data comprising the text-based input data. Alternatively or in addition, obtaining textual data may comprise retrieving the text-based input data from a data storage, such as the subject's EHR or other comparable source. The input-data text data may comprise structured and/or unstructured text.

As an example, pre-processing the text-based input data may comprise detecting at least an abbreviation in the text-based input data, and, optionally replacing the abbreviation with a respective expression. In particular, pre-processing the text-based input data may comprise deriving the at least one textual element from the textual data comprising the respective expression. For example, the pre-processing may involve inputting the expression into the machine learning model as part of the textual data. In particular, pre-processing the text-based input data may comprise detecting the abbreviation in the text-based input data and generating the textual data, wherein in the textual data the abbreviation is replaced by the respective expression. In particular, replacing the abbreviation with the respective expression may be rule-based. More particularly, replacing the abbreviation with the respective expression may be carried out according to a rule, the rule associating the abbreviation with the respective expression. In particular, pre-processing the text-based input data may comprise accessing said rule.

For instance, the text-based input data may comprise the alphanumeric string: “AP 11-w intrauterine pregnancy”. In this case, the abbreviation detected may be the alphanumeric string “AP” and the respective expression may be the respective string “abdominal pain”. Hence, in this case the textual data, generated from the text-based input data, comprises the alphanumeric string: “Abdominal pain 11-w intrauterine pregnancy”.

Exemplarily, pre-processing the text-based input data, e.g., replacing the abbreviation with the respective expression and/or deriving the textual element from the textual data comprising the respective expression, can be rule-based and/or AI-supported.

An AI-supported expansion may comprise inputting the text-based input data or a part thereof, said part comprising the abbreviation, into a model that is trained for detecting abbreviations and replacing the detected abbreviations with respective expressions.

Optionally, a plurality of abbreviations may be detected in the text-based input data and each abbreviation of the plurality of abbreviations may be replaced with a respective expression. In particular, the replacement of the abbreviations may be rule-based. More particularly, pre-processing the text-based input data may comprise accessing a set of rules e.g., comprised in a dictionary, wherein each rule of the set of rules associating a respective abbreviation of a set of abbreviations to a respective expression. In this case, in particular, pre-processing the text-based input data may comprise accessing the set of rules to determine abbreviations of the set of abbreviations, detecting said abbreviations in the input-based text, and replacing the detected abbreviation with corresponding expression according to the rules of the set of rules, thereby obtaining the textual data. For instance, the set of abbreviations may comprise health state related abbreviations.

Alternatively, or in addition, pre-processing the text-based input data may comprise detecting a portion of the text-based input data that matches an abbreviation (herein also referred to as: “matched abbreviation”) among a plurality of abbreviations. Said detection may be based on a similarity measure so that the matching abbreviation is the abbreviation of the plurality of abbreviations that is most similar to the portion of the text-based input data. For instance, pre-processing the text-based input data may comprise selecting an expression to replace the portion of the text-based input data based on the matching abbreviation and replacing that portion with that respective expression.

Pre-processing the text-based input data may comprise detecting and correcting typographical errors and/or spelling errors. This can be done using the same or similar steps as described in the context of an abbreviation. In particular, the steps described above for replacing an abbreviation with a respective expression may be carried out, mutatis mutandis, for replacing a text comprising typographical errors and/or a spelling error with a correct text.

The method of the present disclosure allows for relatively few restrictions on the form and content of the input data. The pre-processing, such as expansion of abbreviations or the correction of errors, increases the flexibility in terms of the input data even further. In many fields, e.g., in the medical field, time-sensitive activities force personnel to take very concise notes, which often includes several abbreviations and/or errors. Pre-processing said notes allows to cope with the presence of abbreviations and/or errors, thereby not decreasing the accuracy of the output obtained by the model of the present disclosure from the notes. Moreover, this makes the method applicable, without loss of accuracy, retrospectively to older user inputs, e.g. retrieved from a data storage, wherein there was lower awareness level of potential automated data analysis, and abbreviations may have been even used more extensively.

According to the present disclosure, deriving the at least one textual element may comprise deriving one or more further textual elements, wherein each further textual element of the one or more further textual elements comprises information indicative of a health state of the subject. In this case, the method may comprise, for each further textual element of the one or more further textual elements: embedding said each further textual element into a respective further second numeric vector by using the embedding model; searching, among the set of first numeric vectors, a respective further closest numeric vector that is closest to said respective further second numeric vector; and determining the health state that is represented by the respective further closest numeric vector as a respective further health state indicated by the further textual element.

In particular, the textual element and the further textual elements may constitute a plurality of textual elements. In this case, exemplarily, the present disclosure refers to a computer implement method comprising:
obtaining a set of first numeric vectors, each first numeric vector representative of a respective health state, the first numeric vectors having been created by using an embedding model;
obtaining textual data, the textual data comprising information indicative of one or more health states of a subject;
using a machine learning model for deriving, from the textual data, a plurality of textual elements, wherein each textual element of the plurality of textual elements comprises information indicative of a respective health state of the one or more health states of the subject.

Moreover, the method may comprise, for each textual element of the plurality of textual elements:
embedding said each textual element into a respective second numeric vector by using the embedding model;
searching, among the set of first numeric vectors, a respective closest first numeric vector that is closest to the second numeric vector; and
determining the respective health state that is represented by the respective closest first numeric vector as a respective health state indicated by said each textual element.

In particular, the method of the present disclosure may comprise generating a plurality of respective health states indicated by the textual elements, each health state of the plurality of respective health states being determined as disclosed above. In particular, the method of the present disclosure as described for a textual element may be carried out accordingly, i.e. in the same manner, for multiple textual elements, e.g., for multiple textual elements derived from the textual data, which e.g., may be a single prompt. Each textual element is embedded into a respective second numeric vector and the closest numeric vector for each said second numeric vectors is searched. That is, the textual elements may be analyzed individually. Thus, multiple health states, each identified by a respective textual element, may be determined from the data input, such as the prompt.

The method may comprise determining an overall health state for the subject, wherein the overall health state comprises information specifying the plurality of health states described above.

An advantage of such a method is that the data input does not need pre-processing that separates the data input into smaller sub-units in advance. A data input that comprises or may potentially comprise data indicative of multiple different health states can be processed. This allows for high flexibility of the method.

According to the present disclosure, the embedding model may comprise or be a first large language model.

Large language model architectures as known in the art may be employed. A large language model, according to the present disclosure, may comprise one or more artificial neural networks utilizing a transformer architecture. Large language model architectures have the ability of natural language processing tasks, e.g. classification and/or language generation, but may also have additional abilities. Moreover, large language models are not necessarily limited to textual input and output, but may be multimodal, e.g. allowing for processing image data, audio data, or the like, optionally both at the input and the output side.

An embedding model is in particular a machine learning model configured to receive a text as input and generate as output a numeric vector which represent that text in a vector space. In particular, the embedding model is configured to map a text into a numeric vector. This mapping is such that a semantic similarity between two texts, as perceived by humans, corresponds to a proximity in the vector space of the vectors representing said texts.

According to the present disclosure, the machine learning model may be a Generative Pre-trained Transformer (GPT) model. GPT models are a type of large language models and are suitable for providing standardized and well-ordered textual output. As an example, from a sentence or a part of a sentence, GPT models can extract keywords, which may already be comprised in the input of the model or derived therefrom. For instance, the output of the GPT model may include the extracted keywords and does not comprise the other words included in the input. GPT models may parse an input into a plurality of textual elements, based on the semantic dissimilarity that said textual elements have with one another. An output may, for example, be in the form of an itemized list, e.g. a list of keywords. Such an output may improve processing by other automated processes, including but not limited to processing by an embedding model. Particularly, when using this type of output as input for an embedding model, resources required for processing this input by the embedding model may be reduced and accuracy increased.

The method may optionally comprise training the machine learning model. If the machine learning model is pre-trained, the method may optionally comprise customizing the machine learning model to the task of deriving, from the textual data, at least one textual element. This customization may be carried out by using transfer learning and/or fine tuning.

According to the present disclosure, deriving the at least one textual element may comprise inserting the textual data into a prompt for the machine learning model. A prompt may comprise text, e.g. natural language text, describing a task that the machine learning model should perform.

Inserting the textual data into the prompt for the machine learning model may comprise inserting text received as part of a user input, e.g. received via a user input device/interface. Alternatively, inserting the textual data into the prompt for the machine learning model may comprise creating the prompt at least by inserting automatically the textual data into the prompt. The prompt may further comprise instructions for processing the textual element. As will be understood from the above, individual textual data will yield an individual prompt. This prompt, accordingly, provides the machine learning model with the specific task at hand, i.e., processing the specific textual data that has been inserted into the prompt. Optionally, a system prompt may also be input into the machine learning model. A system prompt may be a generalized prompt that may be applied for each task at hand. That is, the system prompt may be independent from the prompt into which the textual data was input, in particular independent from the specific task at hand. A system prompt may be a prompt that comprises general instructions for processing textual elements. A system prompt may comprise instructions, context information, and/or guidelines that are provided to the machine learning model separately from, in particularly prior to, processing a prompt comprising the textual element, such as a prompt input by a user. For example, system prompts may provide a framework and/or parameters guiding the machine learning model's processing of a prompt comprising the textual element, such as a prompt input by a user.

Inserting the textual data into the prompt allows for avoiding any need for a special API or other type of specialized interface for inputting and processing the textual data. As such, the proposed feature provides a solution that is technically robust and versatile in its application. For example, an end user may directly enter and/or select textual data without any special hardware or software equipment.

Exemplarily, creating the set of first numeric vectors may comprise, for each first numeric vector of the set of first numeric vectors, embedding a respective, particularly textual, representation of the respective health state into said each first numeric vector by using the embedding model.

In particular, for each health state, the respective first numeric vector is obtained by selecting a respective reference textual representation of the health state and by embedding that reference textual representation into the respective first numeric vector. To that end, the respective reference textual representation of the health state may be input into the embedding model. For instance, if the health state is pregnancy, the respective reference textual representation may be the word “pregnancy”, the word “gravidity”, or the abbreviation “IUP”. The embedding model may be configured to output the first numeric vector(s) and/or use the first numeric vector(s) for further steps, such as for identifying closest numeric vectors.

According to the present disclosure, the embedding model may be configured to map the at least one textual element to one or more of the health states.

In particular, the embedding model may be configured to map the textual element to the health state associated with the first numeric vector that is closest to the second numeric vector. For instance, the embedding model is at least configured to: (i) embedding the textual element into the second numeric vector; (ii) searching, among the set of first numeric vectors, the closest numeric vector that is closest to the second numeric vector; and (iii) determining the health state that is represented by the closest numeric vector as a health state indicated by the textual element. In particular, multiple steps of the present disclosure may be carried out by means of the embedding model. In particular, in addition to any embedding steps, the embedding model may be configured to also perform the searching, among the set of first numeric vectors, the closest numeric vector that is closest to the second numeric vector and the determining the health state that is represented by the closest numeric vector as a health state indicated by the textual element.

Exemplarily, the health states may be standardized chief complaints or standardized categories of chief complaints. Each textual element may be representative of a respective chief complaint

As an example, a standardized category of chief complaints may be “pregnancy” and it may be representative of several patient complaints, such as “11 week intrauterine pregnancy”, “15 week pregnancy”, “intrauterine pregnancy” etc., which are all in the “pregnancy” category.

The method of the present disclosure may further comprise predicting a future health state of the subject by using the health state indicated by the textual element via a prediction algorithm. In particular, the prediction algorithm may be a machine learning model that can be trained on retrospective data of subjects' EHR data. The input of the prediction algorithm can comprise information about the health of a respective subject. for instance, the input may comprise one or more of: one or more health states of the subject (e.g., one or more chief complaints of the subject), demographic data (e.g., age, gender and the like), medical history of the subject, vitals available at ED triage time. The target function for such a prediction algorithm may provide as output the type of hospital resource needed for the subject For example, the hospital resource may be ICU, inpatient hospitalization, and/or urgent procedures. This prediction algorithm can then be used to optimize triage decisions on a subject by providing an acuity score that reflects an expected need for any specific type of hospital resources.

For example, the prediction algorithm may be configured to determine, based on known prediction methods, expected changes in the overall health state of the subject and/or in one of the determined health states of the subject.

As already mentioned above, identifying a health state of the subject according to the method of the present disclosure may allow for using the output for further automated processing. In particular, the prediction of a future health state may be an outcome of this automated processing. This way, patient care can be made even more reliable, particularly concerning expected future needs, in addition to present needs. Thus, resources can be made available accordingly to improve overall outcomes.

Generally, according to the present disclosure, a set may comprise one or more elements. Accordingly, the set of first numeric vectors comprises at least one first numeric vector, i.e. one or more first numeric vectors. Exemplarily, the set of first numeric vectors may comprise a plurality of first numeric vectors.

A further aspect of the present disclosure relates to a system comprising a processing system configured to obtain a set of first numeric vectors, each first numeric vector representative of a respective health state, the first numeric vectors having been created by using an embedding model; obtain textual data, the textual data comprising information indicative of one or more health states of a subject; use a machine learning model for deriving, from the textual data, at least one textual element, wherein the textual element comprises information indicative of a health state of the one or more health states of the subject; embedding the at least one textual element into a second numeric vector by using the embedding model; search, among the set of first numeric vectors, a closest numeric vector that is closest to the second numeric vector; and determine the health state that is represented by the closest numeric vector as a health state of the subject represented by the textual element.

Exemplarily, the system, particularly the processing system comprised in the system, is configured to carry out the method according to the present disclosure, as described hereinabove and hereinbelow. Generally, the system may be fully or partially part of a clinical decision support system or in communicative connection with a clinical decision support system.

The system, e.g., the processing system comprised in the system, may comprise a user input device. For instance, the user input device may be configured to receive textual input data.

In particular, the processing system is a computing device. A computing device may comprise at least one memory and at least one processor. A computing device may also comprise one or more input/output units. A computing device may be a single device or comprises multiple devices, such as in a distributed processing system. For example, according to the present disclosure, a computing device may be any type of data processing device, such as a smartphone, a desktop computer, a server, a server network, a cloud computing network, or the like. According to the present disclosure, a computing device may include one or more processors for data processing and at least one data storage for storing data, such as data representing the health states, the first numeric vectors, the input data, such as clinical report or patient record, or the like.

Alternatively or additionally, a computer program or software instructions may be stored on the data storage, which, when executed by one or more processors of the computing device, instructs the processing system (computing device) to perform steps of the method according to the present disclosure, as described hereinabove and hereinbelow.

Optionally, the computing device may comprise at least one communication circuitry or interface for communicatively coupling the computing device to one or more external data sources that may optionally store data.

According to a further aspect of the present disclosure, there is provided a computer program product, which, when executed by a computer, e.g. by one or more processors of a computing device, cause the computer to carry out the steps of the method according to the present disclosure, as described hereinabove and hereinbelow.

According to a further aspect of the present disclosure, there is provided a computer-readable medium, e.g., a non-transitory computer-readable medium, storing instructions, such as a computer program, which, when executed by a computer, e.g. by one or more processors of a computing device, cause the computer to carry out the steps of the method according to the present disclosure, as described hereinabove and hereinbelow.

Features and advantages described herein in the context of the computer-implemented method apply accordingly also to the computer program product, computer-implemented method, and processing system.

These and other aspects of the disclosure will be apparent from and elucidated with reference to the appended figures, which may represent exemplary embodiments.

### BRIEF DESCRIPTION OF THE DRAWINGS

The subject-matter of the present disclosure will be explained in more detail in the following with reference to examples and embodiments of the present disclosure which are illustrated in the attached drawings, wherein:

FIG. 1 shows a processing system according to the present disclosure; and

FIG. 2 shows a flow chart illustrating a method according to the present disclosure; and

FIG. 3 shows an exemplary illustration of the method of the present disclosure and potential data flow.

The figures are schematic only and not true to scale.

### DETAILED DESCRIPTION

The present disclosure provides a system 1 comprising a processing system 100 configured to obtain a set of first numeric vectors, each first numeric vector representative of a respective health state, the first numeric vectors having been created by using an embedding model; obtain textual data, the textual data comprising information indicative of one or more health states of a subject; use a machine learning model for deriving, from the textual data, at least one textual element, wherein the textual element comprises information indicative of at least one health state of the one or more health states of the subject; embedding the at least one textual element into a second numeric vector by using the embedding model; search, among the set of first numeric vectors, a closest numeric vector that is closest to the second numeric vector; and determine the health state that is represented by the closest numeric vector as a health state of the subject represented by the textual element. In particular, exemplarily, the system 1, particularly the processing system 100 comprised in the system 1, is configured to carry out the method according to the present disclosure, as described hereinabove and hereinbelow.

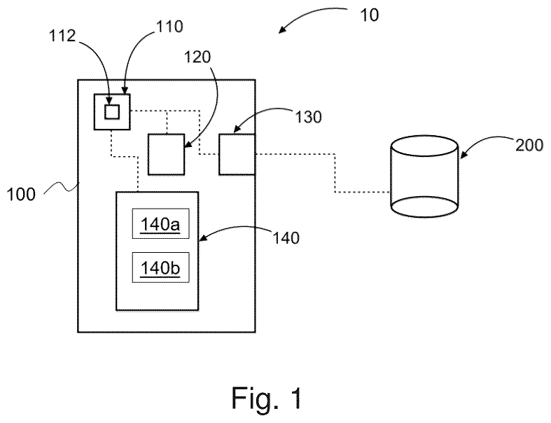

FIG. 1 shows an example of how said system 1 comprising the processing system 100, referred to as computing device hereinbelow, according to the present disclosure may be configured. It is to be understood that other configurations are also possible.

As an illustrative example, the computing device 100 may comprise a processing circuitry 110 or control circuitry 110 with one or more processors 112 for data processing. Optionally, the processing circuitry 110 or control circuitry 110 may include a classifier or a classifier circuitry. The computing device 100 further comprises at least one data storage 120 for storing data.

In this illustrative example, the computing device 100 of FIG. 1 further comprises at least one communication circuitry or interface 130 for communicatively coupling the computing device 100 to one or more external data sources 200 that may optionally store data and/or provide data to the computing device 100. The communication circuitry or interface 130 may be configured for wired or wireless communication with the at least one external data source 200. It should be noted that the computing device 100 may comprise a plurality of communication circuits or interfaces 130 for communicatively coupling the computing device 100 to a plurality of different external data sources 200.

The one or more external data sources 200 may for example be associated with one or more external servers communicatively coupled to the computing device 100, for example via the Internet, a LAN connection, a wireless connection or a wired connection. For example, the computing device 100 may be communicatively couplable to a hospital information system (not shown), a laboratory information system (not shown), a server of a health care provider (not shown), or any other server.

Optionally, the computing device 100 may be configured to obtain, e.g., retrieve data (e.g., the textual data, the text-based input, the health states and/or the set of first numeric vectors), from a data source external to the computing device 100.

The computing device 100 may include a user interface 140 for receiving one or more user inputs, the user interface being or comprising a user input device 140a. For instance, input data may be provided to the computing device 100 via the user interface 140, particularly via the user input device 140a. Said input data may for instance comprise or consist of the textual data, or of the text-based input. Said input data may comprise textual input data to be used for obtaining the textual data, e.g. said input data may comprise at least a portion of a medical report.

The user interface 140 may be configured to provide or output information to a user, for example via a display portion 140b of a display device. For example, the computing device 100 may be configured to display the health state indicated by the textual element and/or a prediction of a future health state at the user interface 140, particularly at a display device and/or display portion 140b.

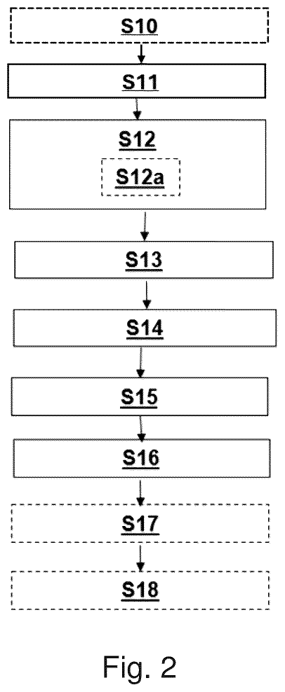

FIG. 2 shows a flow chart illustrating a computer-implemented method according to an exemplary embodiment of the present disclosure. The method may, for example, be carried out by the computing device 100 as described with reference to FIG. 1 or other suitable computing devices.

Step S11 comprises obtaining a set of first numeric vectors, each first numeric vector representative of a respective health state, the first numeric vectors having been created by using an embedding model. In particular, at least some of the health states may be standardized chief complaints or standardized categories of chief complaints.

The embedding model may comprise or consist of a first large language model. The embedding model may be configured to process at least text input. It may be configured to also process other type of input, i.e., a mixed-type input, such as text input, image input or the like.

At step S12, the computing device obtains textual data, the textual data comprising information indicative of one or more health states of a subject. Optionally, step S12 may comprise pre-processing text-based input data to obtain the textual data S12a. As an example, pre-processing the text-based input data may comprise detecting at least an abbreviation in the text-based input data, optionally replacing the abbreviation with a respective expression, and deriving the at least one textual element from the textual data comprising the respective expression. For this example, pre-processing the text-based input data, replacing the abbreviation with a respective expression, and/or deriving the textual element from the at least one detected abbreviation may be rule-based and/or AI-supported.

Step S13 comprises using a machine learning model for deriving, from the textual data, at least one textual element, wherein the textual element comprises information indicative of a state of the one or more health states of the subject.

The machine learning model may comprise or consist of one or more GPT models. For example, the machine learning model may be ChatGPT. Deriving the at least one textual element may comprise inserting the textual data into a prompt for the machine learning model. In particular, the textual data may, for example, be input as a prompt or as part of a prompt to the machine learning model. Said inputting may be carried out by ways of receiving a user input and/or automatically, e.g. by automatically receiving and/or retrieving data from a data source and inputting it into the prompt.

Exemplarily, Step S13 may comprise using the machine learning model for deriving, from the textual data, a plurality of textual elements, wherein each textual element of the plurality of textual elements comprises information indicative of a respective health state of the one or more health states of the subject. In particular, in this case, step S13 allows for deriving multiple textual elements from the textual data, e.g. from a single input such as a prompt.

Step S14 comprises embedding the at least one textual element into a second numeric vector by using the embedding model. If multiple textual elements are derived in step S13, step S14 may comprise, for each textual element of the plurality of textual elements, embedding that textual element into a respective second numeric vector by using the embedding model.

Step S15 comprises searching, among the set of first numeric vectors, a closest numeric vector that is closest to the second numeric vector. If multiple textual elements are derived in step S13, step S15 may comprise, for each textual element of the plurality of textual elements, searching, among the set of first numeric vectors, a respective closest first numeric vector that is closest to the respective second numeric vector into which that textual element is embedded.

The closest numeric vector(s) can be determined by any suitable distance metric. The distance may for example be the cosine distance, the Euclidean distance, the Manhattan distance, the Chebyshev distance, the Hamming distance, or the like.

Step S16 comprises determining the health state that is represented by the closest numeric vector as a health state indicated by the textual element. If multiple textual elements are derived in step S13, step S16 may comprise, for each textual element of the plurality of textual elements, determining the respective health state that is represented by the respective closest first numeric vector as a respective health state indicated by said each textual element. If multiple textual elements are derived in step S13, step S16 generates a plurality of respective health states indicated by the textual elements, each health state of the plurality of respective health states being determined as disclosed above.

According to the method of the present disclosure, each textual element may be representative of a respective chief complaint of the subject, and the embedding model may be configured to map that respective chief complaint to one standardized chief complaint of one or more standardized chief complaints or to one standardized category of chief complaints of one or more standardized categories of chief complaints.

Optionally, the method schematically depicted in FIG. 2 may comprise generating the set of first numeric vectors S10. In particular, step S10 comprises, for each health state of a plurality of health states, embedding a respective, particularly textual, representation of the respective health state into a respective first numeric vector of the set of first numeric vectors, said vector being representative of said each health state.

In optional step S17, the health state or the plurality of plurality of respective health states determined in step S16 may be output by a display device and/or used as input for further determination steps, such as predictive steps.

An optional exemplary predictive step is step S18, which comprises predicting, via a prediction algorithm, a future health state of the subject by using the health state(s) indicated by the textual element(s).

Further examples, embodiments, and advantages of the method and system of the present disclosure will become apparent from the following discussion.

CDS applications depend on precise chief complaints mapping to generate accurate risk scores. Complaints are often stored in EHR systems as unstructured text or customizable categories. This contributes to inaccurate complaint mapping that can result in erroneous risk scores. Present chief complaint mapping is time consuming and inaccurate.

The present disclosure addresses these issues and discloses methods and systems that may use Generative AI techniques to significantly enhance complaint mapping accuracy. This will improve clinical performance and increase the adoption of CDS application to improve risk score predictions and, ultimately, patient care.

Techniques, as disclosed by the present disclosure, can significantly enhance the mapping process of patient complaints into structured categories. This enhancement will bridge the gap between the often-unstructured patient complaints and the structured inputs typically required by CDS applications.

Thus, the present disclosure directly addresses key operational challenges in healthcare technology deployment, such as the reliability of chief complaint mapping. For example, by automating the complaint mapping process with advanced AI, the present method and system achieve higher, e.g. 250% higher, accuracy than conventional mapping methodologies, thereby reducing the potential for problems stemming from data inaccuracies. This enhances the performance of downstream machine learning tools and greatly streamlines the installation process. Hospitals and healthcare facilities can now integrate this technology faster.

Typically, in conventional methods, the assessment and proper categorization of patient complaints may take up to 45 hours per hospital site. With the proposed system and method, this time invest can be reduced significantly, e.g. to less than one hour. Consequently, considering the use of this CDS method at a plurality of hospital sites, savings of several thousand hours of time could be achieved per year. Similarly, the time that is presently invested in updating and maintaining the functionality of conventional CDS installations may be reduced by several hours per year and site.

By more accurately translating unstructured or even partially incomplete patient complaints into structured categories the present disclosure enables connected CDS tools to more accurately predict adverse events for a subject and this, ultimately, facilitates better patient care outcomes because medical practitioners are made aware of potentially critical patient needs much earlier.

As an example, electronic health records (EHR) systems like EPIC and CERNER often allow for unstructured text entries or customizable categories, complicating the deployment of a standardized CDS product across different hospitals. This variability significantly impacts the effectiveness of all CDS application which require a precise categorization of patient complaints to provide time and accurate guidance regarding the health situation of assessed patients. Discrepancies in the complaint assessment or a wrong complaint categorization may lead to erroneous outcomes and may negatively affect the quality of subsequent patient care.

The present disclosure leverages two machine learning models and utilizes the capabilities of generative AI. For example two Large Language Models (LLMs) may be combined to accurately identify an individual patients chief patient. The more accurately identified chief complaints of a patient may then be used as input variables for a further tool, ideally another ML based CDS tool, to assess each patient's specific need for care, e.g. from health care professionals in an emergency department setting. The resulting quick and accurate patient triage will lead to a faster and more appropriate patient treatment and will, ultimately, improve patient health. Thus, the present disclosure represents a significant step forward in healthcare technology.

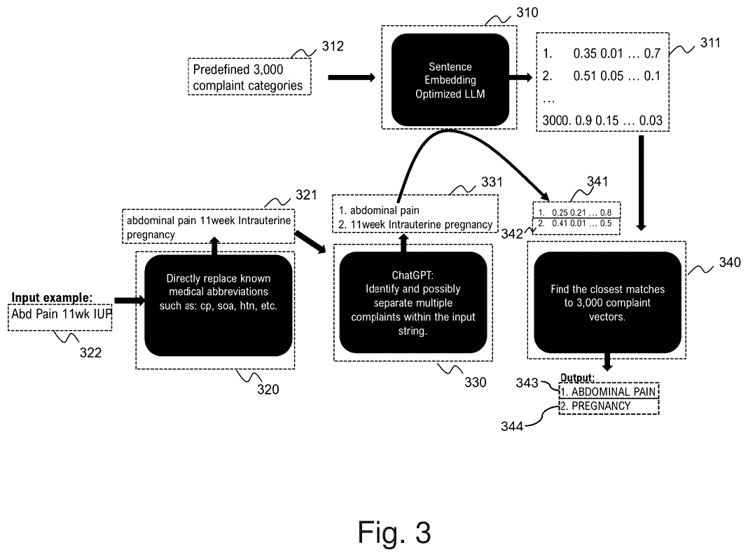

FIG. 3 illustrates an example workflow according to the present disclosure.

Block 310 schematically depicts a non-limiting example for optional step S10 of creating the set of first numerical vectors. In particular, step S10 may comprise, for each health state of a plurality of health states, embedding a respective, particularly textual, representation of the respective health state into a respective first numeric vector of the set of first numerical sectors, said vector being representative of said each health state, by using the embedding model.

For example, a predefined list of N chief complaint categories 312, is mapped into a corresponding set 311 of N numerical vectors. For example, N is an integer that may be equal to 3,000. In particular, this mapping may be carried out using the embedding model, such as a Large Language Model (LLM), which is configured to map conceptually similar sentences close to each other in a vector space.

The embedding of the chief complaint categories may be carried one once. It is noted, however, that the embedding step may be carried out multiple times, as an example, to update the list of complaint categories and corresponding vectors.

Blocks 320, 330 and 340 describe steps that are carried out in response of providing, e.g., by a nurse or a physician, the text-based input to the computing device, e.g., by using the user input device 140a. the text-based input may in particular be unstructured text that may comprise abbreviation, typographical errors and/or spelling errors. As shown in FIG. 3, for example, the text-based input may consist of the exemplary input “Abd Pain 11 wk IUP” 322.

At block 320, the computing device accesses and pre-processes the text-based input to generate the textual data. In particular the computing device 100 detects abbreviations in the text-based input and replaces the detected abbreviations according to a set of rules associating medical abbreviations with respective textual expressions. For instance, the set of rules may comprise a rule indicating to replace the abbreviation “abd” with the expression “abdominal”, a rule indicating to replace the abbreviation “wk” with the expression “week”, a rule to replace the abbreviation “IUP” with the expression “Intrauterine pregnancy”. For instance, pre-processing the text-based input “Abd Pain 11 wk IUP” 322 as described above leads to the textual data “abdominal pain 11 week Intrauterine pregnancy” 321.

At block 330, one or more GPT models, e.g., ChatGPT, is used, optionally together with suitable prompt engineering, to derive one or more textual elements from the textual data, each textual element of the one or more textual elements comprising information indicative of a respective health state of the subject.

The one or more textual elements derived by the one or more GPT models may be cast in an itemized list, wherein each item of the itemized list corresponds to a respective textual element of the one or more textual elements. For example, if the textual data is the alphanumeric string “abdominal pain 11 week Intrauterine pregnancy” 321 the one or more textual elements are “abdominal pain” and “11 week Intrauterine pregnancy”, which are cast in the itemized list 331.

Moreover, for each textual element of the one or more of textual elements, that textual element is embedded into a respective second numeric vector by using the embedding model. For example, with reference to FIG. 3, the textual element “abdominal pain” is embedded into the numeric vector (0.25, 0.21, . . . , 0.8) 341 and the textual element “11 week Intrauterine pregnancy” is embedded into the numeric vector (0.41, 0.01, . . . , 0.5) 342.

At block 340, for each textual element of the plurality of textual elements, it is searched, among the set of first numeric vectors, a respective closest first numeric vector that is closest to the respective second numeric vector into which that textual element is embedded and it is determined the respective health state that is represented by the respective closest first numeric vector as a respective health state indicated by that textual element. In particular, one or more respective health states indicated by the textual elements are generated, each health state of the plurality of respective health states being determined as disclosed above. In particular, in this case, the plurality of respective health states constitutes the output of block 340. For example, if the one or more textual elements are abdominal pain” and “11 week Intrauterine pregnancy” the output consist of the chief complaint “ABDOMINAL PAIN” 343, associated with the former textual element, and of the chief complaint “PREGNANCY” 344 associated with the latter textual element.

Each raw in the table below collects an exemplary text-based input and the corresponding output of block 340, wherein the output is obtained from the corresponding text based input as schematically shown in FIG. 3 and herein described:

| Text-based input | Output |
|---|---|
| Cannot urinate | 1. URINARY RETENTION |
| since yesterday | |
| Diarrhea Possible | 1. DIARRHEA |
| Syncope Dementia | 2. SYNCOPE |
| nausea | 3. DEMENTIA |
| | 4. NAUSEA |
| meth | 1. DRUG ABUSE |
| drunk at lake | 1. ALCOHOLISM |

While the present disclosure has been illustrated and described in detail in the drawings and foregoing description, such illustration and description are to be considered illustrative or exemplary and not restrictive; the disclosure is not limited to the disclosed embodiments. Other variations to the disclosed embodiments can be understood and effected by those skilled in the art and practicing the claimed invention, from a study of the drawings, the disclosure, and the appended claims.

In the claims, the word “comprising” does not exclude other elements or steps, and the indefinite article “a” or “an” does not exclude a plurality. The mere fact that certain measures are recited in mutually different dependent claims does not indicate that a combination of these measures cannot be used to advantage. Any reference signs in the claims should not be construed as limiting the scope.

1. A computer-implemented method comprising:
obtaining a set of first numeric vectors, each first numeric vector representative of a respective health state, the first numeric vectors having been created by using an embedding model;
obtaining textual data, the textual data comprising information indicative of one or more health states of a subject;
using a machine learning model for deriving, from the textual data, at least one textual element, wherein the textual element comprises information indicative of a health state of the one or more health states of the subject;
embedding the at least one textual element into a second numeric vector by using the embedding model;
searching, among the set of first numeric vectors, a closest numeric vector that is closest to the second numeric vector; and
determining the health state that is represented by the closest numeric vector as a health state indicated by the textual element.

2. The method of claim 1, wherein, obtaining textual data comprises pre-processing (S12a) text-based input data to obtain the textual data.

3. The method of claim 2, wherein pre-processing the text-based input data comprises detecting at least an abbreviation in the text-based input data, optionally replacing the abbreviation with a respective expression, and deriving the at least one textual element from the textual data comprising the respective expression.

4. The method of claim 3, wherein pre-processing the text-based input data, replacing the abbreviation with a respective expression, and/or deriving the textual element from the at least one detected abbreviation is rule-based and/or AI-supported.

5. The method of claim 1,
wherein deriving the at least one textual element comprises deriving one or more further textual elements, wherein each further textual element of the one or more further textual elements comprises information indicative of a health state of the subject,
wherein, for each further textual element of the one or more further textual elements, the method further comprises:
embedding said each further textual element into a respective further second numeric vector by using the embedding model;
searching, among the set of first numeric vectors, a respective further closest numeric vector that is closest to said respective further second numeric vector; and
determining the health state that is represented by the respective further closest numeric vector as a respective further health state indicated by the further textual element.

6. The method of claim 1, wherein the embedding model comprises or is a first large language model.

7. The method of claim 1, wherein the machine learning model is a generative pre-trained transformer model.

8. The method of claim 1, wherein deriving the at least one textual element comprises inserting the textual data into a prompt for the machine learning model.

9. The method of claim 1, wherein creating the set of first numeric vectors comprises, for each first numeric vector of the set of first numeric vectors, embedding a respective, particularly textual, representation of the respective health state into said each first numeric vector by using the embedding model.

10. The method of claim 1, wherein the embedding model is configured to map the at least one textual element to one or more of the health states.

11. The method of claim 10, wherein the health states are standardized chief complaints or standardized categories of chief complaints, wherein each textual element is representative of a respective chief complaint, the machine learning model mapping each respective chief complaint to one of the standardized patient complaints or standardized categories of patient complaints.

12. The method of claim 10, further comprising predicting a future health state of the subject by using the health state indicated by the textual element via a prediction algorithm.

13. A system comprising a processing system configured to:
obtain a set of first numeric vectors, each first numeric vector representative of a respective health state, the first numeric vectors having been created by using an embedding model;
obtain textual data, the textual data comprising information indicative of one or more health states of a subject;
use a machine learning model for deriving, from the textual data, at least one textual element, wherein the textual element comprises information indicative of a state of the one or more health states of the subject;
embedding the at least one textual element into a second numeric vector by using the embedding model;
search, among the set of first numeric vectors, a closest numeric vector that is closest to the second numeric vector; and
determine the health state that is represented by the closest numeric vector as a health state of the subject represented by the textual element.

14. The system of claim 13, configured to carry out the method.

15. The system of claim 13, further comprising a user input device configured to receive textual input data.

16. A computer-program product comprising instructions which, when the program is executed by a computer, cause the computer to carry out the method of claim 1.

17. A computer-readable medium comprising instructions which, when the program is executed by a computer, cause the computer to carry out the method of claim 1.
